(19) United States
(12) Patent Application Publication
(10) Pub. No.: US 2020/0014736 A1
(43) Pub. Date: Jan. 9, 2020

(54) INFORMATION PROCESSING SERVER AND RECORDING MEDIUM

(71) Applicant: Konica Minolta, Inc., Tokyo (JP)

(72) Inventors: Kanako TAKESUE, Tokyo (JP); Wataru MOTOKI, Tokyo (JP); Youko KOUDA, Tokyo (JP)

(21) Appl. No.: 16/454,658

(22) Filed: Jun. 27, 2019

(30) Foreign Application Priority Data
Jul. 9, 2018 (JP) 2018-129844

(51) Int. Cl. H04L 29/06

(52) U.S. Cl. CPC H04L 65/4015; H04L 65/1093; G16H 30/20; H04L 65/403; H04L 65/1089

(57) ABSTRACT

An information processing server to which terminal devices used by respective participants are connected for data communication provides the terminal devices with medical information including a medical image. The information processing server includes a memory and a hardware. The memory includes a shared work area for displaying a shared screen which is commonly shown in the terminal devices and personal work areas which are prepared for the respective participants and which are each for displaying a personal work screen in each of the terminal devices used by the respective participants. The hardware processor provides, according to an operation command from the each of the terminal devices used by the respective participants, the terminal devices used by the respective participants with a processing result of the medical information processed with a predetermined application program, using the personal work areas for the respective participants using the respective terminal devices.

### CROSS REFERENCE TO RELATED APPLICATION

The present application claims priority under 35 U.S.C. § 119 to Japanese Patent Application No. 2018-129844, filed on Jul. 9, 2018, the entire content of which is incorporated herein by reference.

### BACKGROUND

### Technological Field

The present invention relates to an information processing server and a recording medium.

### Description of the Related Art

In recent years, there have been web conference systems which provide a shared screen equally displayed on multiple terminal devices, to be used in conferences or educational occasions. The conference participants may listen to the presenter or exchange opinions while looking through the material(s) shown on the shared screen in their own terminal devices.

Concerning electronic communication systems in which multiple terminal devices are connected on a network and users refer to a common material(s) displayed in each terminal device in a cooperative work, a technique to display a cooperative work space and a personal work space in combination is introduced (see JP2002-051317A).

Materials used in a conference or an educational occasion are usually distributed on the spot or beforehand. In an online conference, participants often download the data of materials beforehand to get ready.

However, in a case where medical images are used as the materials, data quantity may be large or even huge depending on the number of the materials. Thus, problematically, the technical environment of the participants are suppressed, as a high load is imposed in data communication or a memory device for data storage is necessary. In a case where the materials contain medical information such as medical images, it may be necessary that the terminal devices which the participants use have an application program to process the medical information. Further, the materials containing medical information need to be handled with the utmost care to hold the confidentiality of personal information, compared to those used in regular conferences or educational occasions.

### SUMMARY

The present invention is conceived in view of such a problem in the conventional techniques as described above, and has an object of providing information for terminal devices used by the participants without changes being made to the environment of the terminal devices.

To achieve at least one of the abovementioned objects, according to a first aspect of the present invention, an information processing server reflecting one aspect of the present invention, to which terminal devices used by respective participants are connected for data communication and which provides the terminal devices with medical information including a medical image, includes:

a memory which includes a shared work area for displaying a shared screen which is commonly shown in the terminal devices and personal work areas each for displaying a personal work screen in each of the terminal devices used by the respective participants, the work areas being prepared for the respective participants; and

a hardware processor which, according to an operation command from the each of the terminal devices used by the respective participants, provides the each of the terminal devices used by the respective participants with a processing result of the medical information processed with a predetermined application program, using the personal work areas for the respective participants using the respective terminal devices.

According to a second aspect of the present invention, a recording medium reflecting one aspect of the present invention stores a program,

wherein the program causes a computer which controls an information processing server to which terminal devices used by respective participants are connected for data communication and which provides the terminal devices with medical information including a medical image:

to prepare, in a memory, a shared work area for displaying a shared screen which is commonly shown in the terminal devices and personal work areas each for displaying a personal work screen in each of the terminal devices used by the respective participants, the work areas being prepared for the respective participants; and

to provide, according to an operation command from the each of the terminal devices used by the respective participants, the each of the terminal devices used by the respective participants with a processing result of the medical information processed with a predetermined application program, using the personal work areas for the respective participants using the respective terminal devices.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 shows a system configuration of a conference system.

FIG. 2 is a block diagram showing a functional configuration of a conference management server.

FIG. 3 shows an example of a data configuration of a host management table.

FIG. 4 shows an example of a data configuration of a conference management table.

FIG. 5 is a block diagram showing a functional configuration of a host terminal and a participant terminal.

FIG. 6 is a ladder chart showing a host registration process.

FIG. 7 is a ladder chart showing a conference setup process.

FIG. 8 shows an example of a host screen displayed in the host terminal.

FIG. 9 shows an example of a conference setup screen displayed in the host terminal.

FIG. 10 is a ladder chart showing a conference organizing process.

FIG. 11 shows an example of a conference screen displayed in the host terminal.

FIG. 12 shows an example of a conference waiting screen displayed in the participant terminal.

FIG. 13 shows an example of a conference screen displayed in the participant terminal.

FIG. 14 shows an example of a personal work screen displayed in the participant terminal.

FIG. 15 is a flow chart showing an initiative transfer process executed in the conference management server.

FIG. 16 is a ladder chart showing a post-conference process (during an available period).

FIG. 17 is a ladder chart showing a post-conference process (outside the available period).

### DETAILED DESCRIPTION OF EMBODIMENTS

Hereinafter, one or more embodiments of the present invention will be described with reference to the drawings. However, the scope of the invention is not limited to the disclosed embodiments.

### Configuration of Conference System

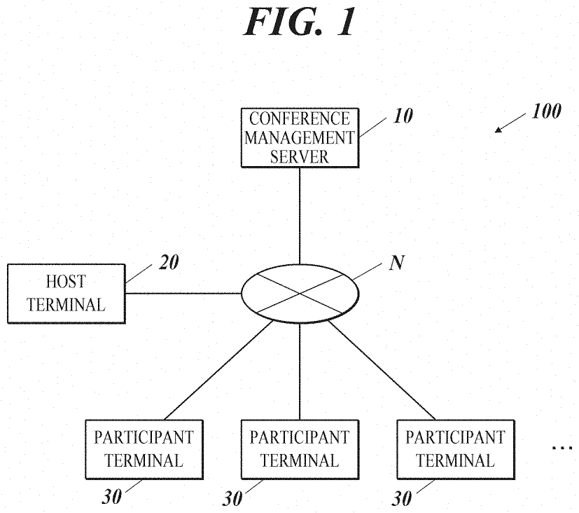

FIG. 1 shows a system configuration of a conference system 100.

As described in FIG. 1, the conference system 100 is configured with a conference management server 10 as an information processing server, a host terminal 20 which is used by a host of the conference, and multiple participant terminals 30 which are respectively used by multiple participants in the conference. The conference management server 10, the host terminal 20, and the participant terminals 30 are connected to each other for data communication via a communication network N. The number of the host terminals 20 and the participant terminals 30 is not particularly limited.

The conference system 100 is used in conferences or educational occasions, where multiple users (host(s) and participant(s)) debate some topics, exchange opinions, or listen to a presentation of a presenter in a study session or a lecture while viewing the shared screens at least.

The conference management server 10 manages information concerning the conference registered via the host terminal 20 and provides medical information containing medical images, etc. for the host terminal 20 and the participant terminal 30. The medical information may include clinical information, disease information, prescription information, examination result data, etc.

The host terminal 20 is a computer device such as a PC (Personal Computer), a tablet terminal, and a smartphone which is used by a host holding the conference.

The participant terminal 30 is a computer device such as a PC, a tablet terminal, and a smart phone which is used by a participant in the conference.

The conference management server 10 described above may be included in a PACS (Picture Archiving and Communication System), an image management device for medical use, as a part of its function. In that case, the PACS may be a cloud-based PACS which may distribute information in the cloud.

### Configuration of Conference Management Server

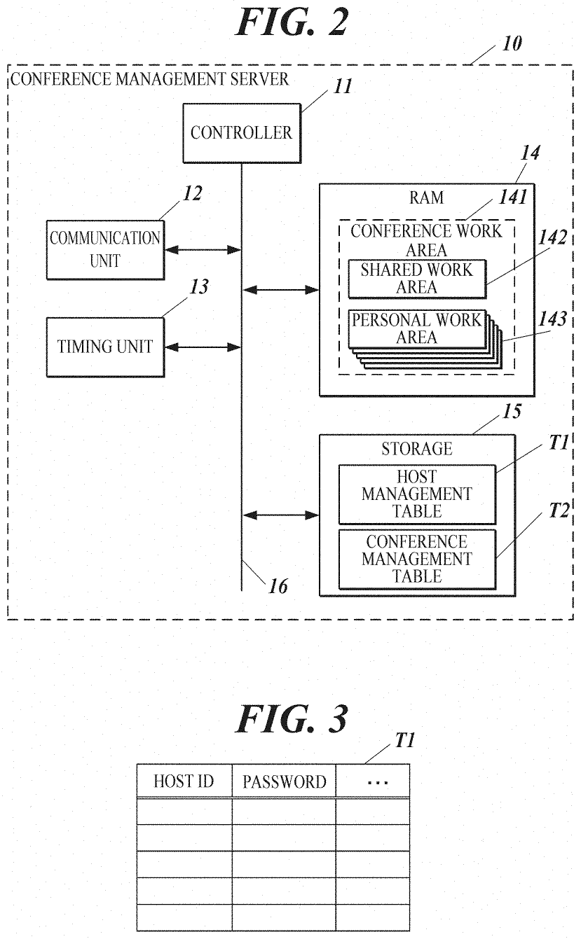

FIG. 2 shows a functional configuration of the conference management server 10. The conference management server 10 is configured with a controller 11, a communication unit 12, a timing unit 13, a RAM (Random Access Memory) 14, a storage 15, etc., which are connected to each other by a bus 16.

The controller 11 is configured with a CPU (Central Processing Unit), etc. and integrally controls the processing operation of each unit of the conference management server 10. Specifically, the CPU reads out various processing programs stored in the storage 15, loads them into the RAM 14, and executes various kinds of processing in cooperation with the programs.

The communication unit 12 is configured with a network interface, etc. and performs data communication with external devices connected via the communication network N such as a LAN (Local Area Network), a WAN (Wide Area Network), and the Internet.

The timing unit 13 has a timing circuit (RTC: Real Time Clock) which measures the current date and time and outputs it to the controller 11.

The RAM 14 is a memory which forms a work area which temporarily stores therein various programs, input/output data, parameters, etc. read out from the storage 15 in the various kinds of processing executed and controlled by the controller 11.

For example, in the RAM 14, a conference work area 141 is provided for each conference to be held. The conference work area 141 includes a shared work area 142 and personal work areas 143 which are each provided for multiple participants. The shared work area 142 is a work area for displaying a shared screen which is common to multiple terminal devices (host terminal(s) 20, participant terminal(s) 30). The personal work area 143 is a work area for displaying a personal work screen on the participant terminal 30 (or host terminal 20) which is used by the participant.

The storage 15 is configured with an HDD (Hard Disk Drive), a non-volatile semi-conductor memory, etc. and stores therein various processing programs, and parameters and files necessary for execution of the programs. For example, the storage 15 stores therein programs such as a web server program(s) to realize a function as a web server which provides various kinds of web screens for a web browser by communicating with the web browser included in the host terminal 20 or the participant terminal 30 by HTTP protocol, and a web application program(s) which works on the web server to provide medical information, etc. for the host terminal 20 and the participant terminal 30 via the web browser. The application program(s) may be, for example, a program to execute display processing, image adjustment processing (adjustment of density, enlargement, reduction, etc.), measurement processing, or annotation addition processing on medical images. The measurement processing is for measurement of distance, angle, ratio, etc. of certain points on a medical image.

The storage 15 stores a host management table T1 and a conference management table T2 therein.

The host management table T1 is a table for management of information concerning a host(s) holding the conference in the conference system 100. As shown in FIG. 3, a host ID, a password, etc. is associated to each host in the host management table T1.

The host ID is identification information (user ID) given to the host.

The password is used for user authentication when the host logs in the system.

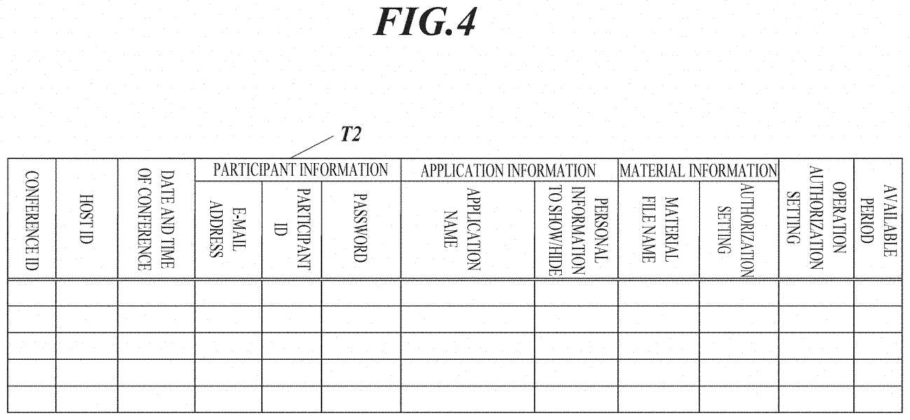

The conference management table T2 is a table for management of information concerning each conference to be held using the conference system 100. As shown in FIG. 4, a conference ID, a host ID, date and time of conference, participant information (e-mail address, participant ID, password), application information (application name, personal information to show/hide), material information (material file name, authorization setting), operation authorization setting, available period, etc. are associated to each conference in the conference management table T2.

The conference ID is identification information given to the conference.

The host ID is a user ID associated to the host holding the conference.

The date and time of conference is date and time when the conference is to be held.

The participant information is information concerning a participant(s) of the conference. The e-mail address is an e-mail address of the participant. The participant ID is identification information (user ID) given to the participant. The password is used for user authentication when the participant logs in the system. Multiple participants may be set in a single conference (conference ID).

The application information is information concerning an application program (hereinafter, it may be referred to just as an “application”) which may be used in the target conference. The application name is a name of the application. The personal information to show/hide is information indicating whether the personal information is to be shown or hidden in the application. Multiple applications may be set in a single conference (conference ID).

The material information is information concerning a material for a conference (medical information, etc.). The material file name is a file name of the material data. The authorization setting is information indicating whether the material associated to the material file name is for viewing (not downloadable) or for distribution (downloadable). Multiple materials may be set in a single conference (conference ID).

The operation authorization setting is information indicating whether an initiative transfer takes place or not. The initiative means authorization for the operation of the shared screen(s).

The available period is a period when the personal work area 143 for each participant in the target conference is available.

The storage 15 stores the material data (medical information such as medical images) associated to the material file name registered in the conference management table T2 therein.

As an account for login (user ID, password) associated to the host or participant is used to access the conference management server 10, the controller 11 refers to the participant information in the host management table T1 and the conference management table T2 in the storage 15, and determines whether it is a user registered beforehand or not. If the combination of the user ID and password input in the host terminal 20 or the participant terminal 30 is registered in the participant information in the host management table T1 or the conference management table T2, the controller 11 determines that the user is an authorized user.

The controller 11 sets the available period concerning the personal work area 143 associated to the target conference according to the operation command from the host terminal 20 used by the host.

The controller 11 provides processing results of the medical information processed by a predetermined application program for the terminal devices, using the personal work area 143 of the participant concerning the terminal device, according to the operation command from the terminal device (participant terminal 30 or host terminal 20) used by each of multiple patients. The controller 11 may use the personal work space 143 during the set available period only.

The personal work screen displayed in the terminal device (participant terminal 30 or host terminal 20) used by a certain participant out of multiple participants is shown on the shared screen common to multiple terminal devices by the controller 11.

### Configuration of Host Terminal

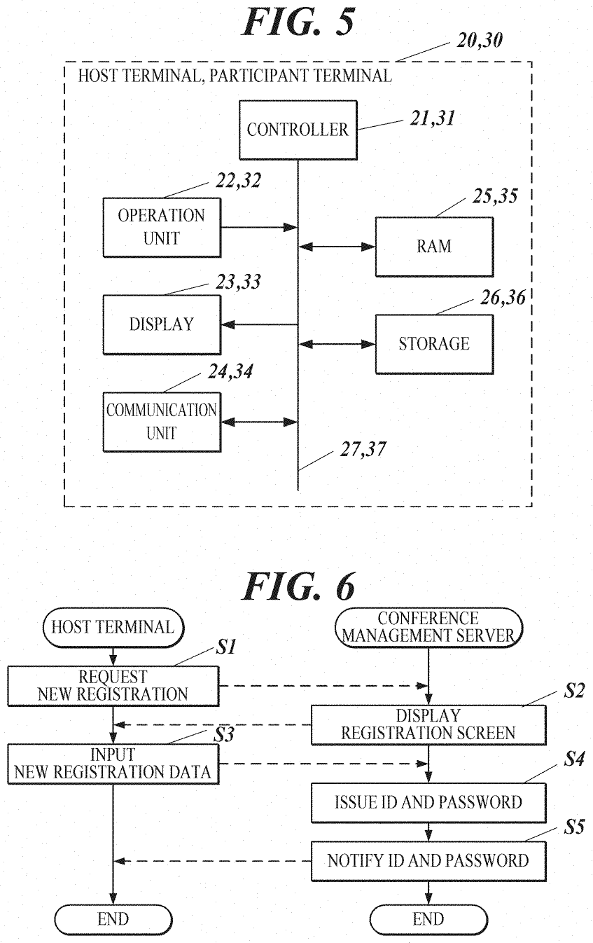

FIG. 5 shows a functional configuration of the host terminal 20. The host terminal 20 is configured with a controller 21, an operation unit 22, a display 23, a communication unit 24, a RAM 25, a storage 26, etc., which are connected to each other by a bus 27.

The controller 21 is configured with a CPU, etc. and integrally controls the processing operation of each unit of the host terminal 20. Specifically, the CPU reads out various processing programs stored in the storage 26, loads them into the RAM 25, and executes various kinds of processing in cooperation with the programs.

The operation unit 22 is configured with a keyboard with cursor keys, letter/number input keys, various function keys, etc. and a pointing device such as a mouse, and outputs, to the controller 21, operation signals input by key operation to the keyboard or the mouse operation. In a case where the operation unit 22 is configured with a touch panel superimposed on the display 23, operation signals according to the position of touch operation with the user's finger, etc. are output to the controller 21.

The display 23 is configured with a monitor such as an LCD (Liquid Crystal Display), and displays various kinds of screens according to the command of display signals input from the controller 21.

The communication unit 24 is configured with a network interface, etc. and performs data communication with external devices connected via the communication network N such as a LAN, a WAN, and the Internet.

The RAM 25 forms a work area which temporarily stores therein various programs, input/output data, parameters, etc. read out from the storage 26 in the various kinds of processing executed and controlled by the controller 21.

The storage 26 is configured with an HDD (Hard disk Drive), a non-volatile semi-conductor memory, etc. and stores therein various processing programs, and parameters and files necessary for execution of the programs. For example, the storage 26 stores therein a web browser program to realize a web browser, etc.

### Configuration of Participant Terminal

FIG. 5 shows a functional configuration of the participant terminal 30. The participant terminal 30 is configured with a controller 31, an operation unit 32, a display 33, a communication unit 34, a RAM 35, a storage 36, etc., which are connected to each other by a bus 37. The configuration of each unit of the participant terminal 30 is not described, as it is the same as that of the host terminal 20.

### Operation in Conference System

Next, the operations in the conference system 100 are described.

### Host Registration Process

FIG. 6 is a ladder chart showing a host registration process executed in the conference system 100.

First, as the host inputs, in the host terminal 20, a URL (Uniform Resource Locator) to access the conference management server 10 on the web browser by the operation via the operation unit 22, the controller 21 requests a new registration to the conference management server 10 via the communication unit 24 using the input URL (Step S1).

In the conference management server 10, the controller 11 sends the data for displaying the registration screen via the communication unit 12 to the host terminal 20 (Step S2). The data for displaying various kinds of web screens such as the registration screen sent to the host terminal 20 by the web server function of the conference management server 10 include an HTML script, a style sheet, image data, a script to execute certain processing in the host terminal 20, etc.

As the host terminal 20 receives the data for displaying the registration screen via the communication unit 24, the registration screen is displayed on the display 23. As the host inputs new registration data (information concerning the host) by the operation via the operation unit 22 (Step S3), the controller 21 sends the input new registration data to the conference management server 10 via the communication unit 24.

In the conference management server 10, the controller 11 issues a host ID (user ID) and password to the newly registered host (Step S4). The controller 11 stores the issued host ID and password in the host management table T1 in the storage 15, associating them to the input new registration data.

The controller 11 notifies the host terminal 20 of the issued host ID and password via the communication unit 12 (Step S5).

The host registration process is completed here.

### Conference Setup Process

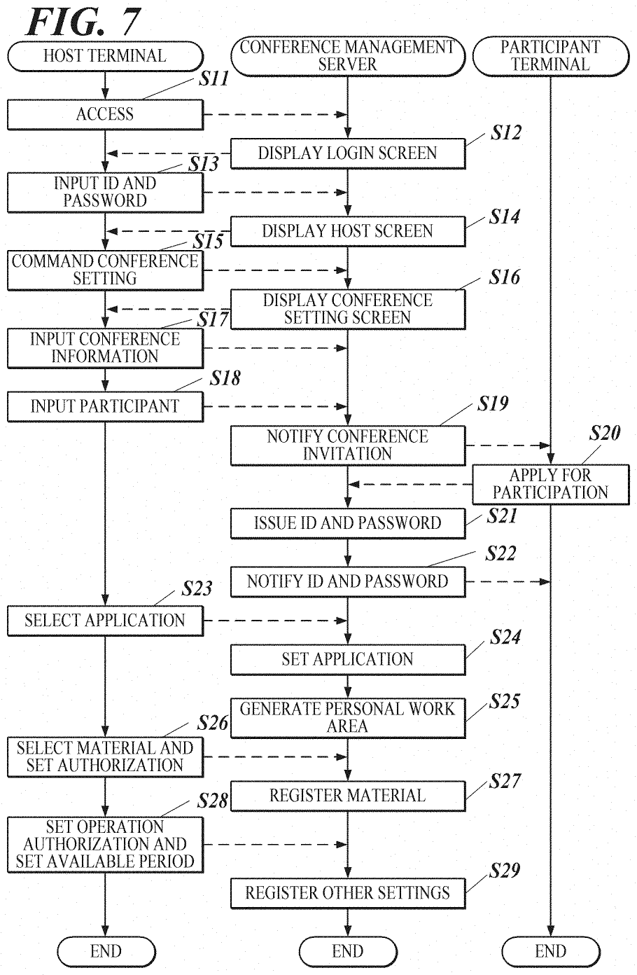

FIG. 7 is a ladder chart showing a conference setup process executed in the conference system 100.

First, as the host accesses the conference management server 10 on the web browser by the operation via the operation unit 22 in the host terminal 20 (Step S11), the controller 11 sends the data for displaying the login screen to the host terminal 20 via the communication unit 12 in the conference management server 10 (Step S12).

The login screen is displayed on the display 23 in the host terminal 20. As the host inputs the user ID (host ID) and password on the login screen by the operation via the operation unit 22 (Step S13), the controller 21 sends the input user ID and password to the conference management server 10 via the communication unit 24.

In the conference management server 10, as the user ID and password are received via the communication unit 12, the controller 11 executes the user authentication. Specifically, the controller 11 retrieves the record corresponding to the user ID input in the host terminal 20 from the host management table T1 in the storage 15. Then, if the password included in the retrieved record corresponds to the password input in the host terminal 20, the controller 11 determines that the user authentication is successfully executed.

If the input user ID is a host ID, the controller 11 sends the data for displaying the host screen to the host terminal 20 via the communication unit 12 (Step S14).

The host screen is displayed on the display 23 in the host terminal 20.

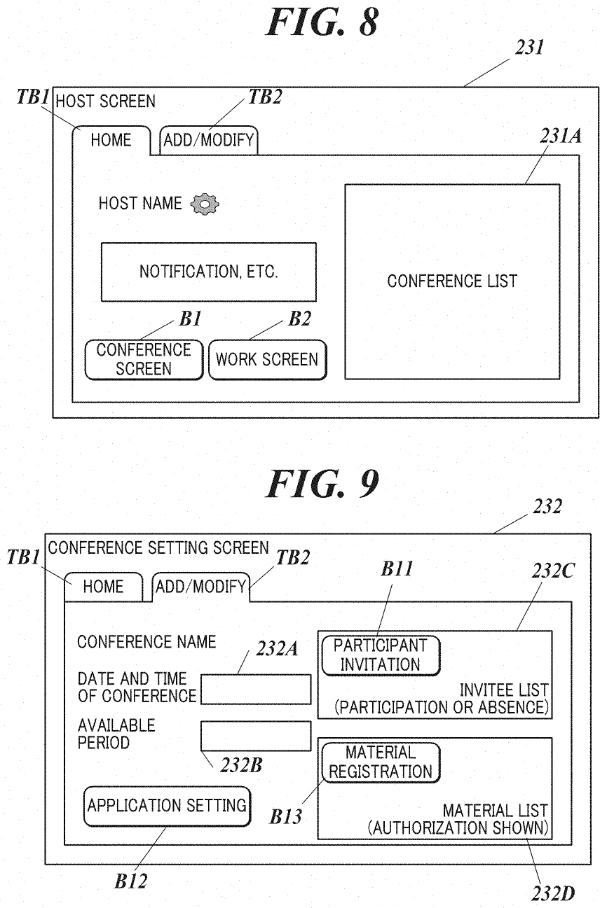

FIG. 8 shows an example of the host screen 231 displayed on the display 23. The host screen 231 includes a “home” tab TB1 and an “add/modify” tab TB2, and the “home” tab TB1 is selected on the host screen 231 in FIG. 8. The host screen 231 includes a conference list display area 231A, a conference screen button B1, a work screen button B2, etc.

The list of the conferences which the host has set up so far is shown in the conference list display area 231A.

The conference screen button B1 is a button for a command of transfer to the conference screen.

The work screen button B2 is a button for a command of transfer to the personal work screen.

The “add/modify” tab TB2 is to be selected when a new conference is set up.

As the host commands a conference setup on the host screen by the operation via the operation unit 22 (Step S15), the controller 11 sends the data for displaying the conference setup screen to the host terminal 20 via the communication unit 12 (Step S16).

Specifically, as the host selects the “add/modify” tab TB2 on the host screen 231 shown in FIG. 8, the conference setup screen 232 shown in FIG. 9 is displayed on the display 23. The conference setup screen 232 includes a date and time input area 232A, an available period input area 232B, an invitee list display area 232C, a material list display area 232D, a participant invitation button B11, an application setting button B12, a material registration button B13, etc.

The date and time when the conference is to be held is input in the date and time input area 232A.

The available period is input in the available period input area 232B.

The list of invitees set for the conference is shown in the invitee list display area 232C. The invitee is an individual who is invited to the conference as a participant by the host. The participation or absence of each invitee is also shown in the invitee list.

The list of the materials set for the conference is shown in the material list display area 232D. The authorization setting determined for each material (downloading allowed or not) is also shown in the material list.

The participant invitation button B11 is a button for a command of transfer to the screen for input of the participant (invitee) in the conference.

The application setting button B12 is a button for a command of transfer to the screen for setting of the application for the conference.

The material registration button B13 is a button for a command of transfer to the screen in which the material for the conference is registered.

As the host inputs the conference information (date and time of the conference, etc.) on the conference setup screen by the operation via the operation unit 22 (Step S17), the controller 21 sends the input conference information to the conference management server 10 via the communication unit 24. Specifically, the host inputs the date and time of the conference in the date and time input area 232A on the conference setup screen 232 shown in FIG. 9.

In the conference management server 10, the controller 11 stores, in the conference management table T2 in the storage 15, the conference information (date and time of the conference, etc.) input in the host terminal 20, associating it to the conference ID and the host ID given to the conference.

Next, as the host presses the participant invitation button B11 on the conference setup screen 232 by the operation via the operation unit 22, the participant input screen is displayed on the display 23. As the host inputs a participant (e-mail address) by the operation via the operation unit 22 (Step S18), the controller 21 sends the input participant to the conference management server 10 via the communication unit 24.

In the conference management server 10, the controller 11 stores, in the conference management table T2 in the storage 15, the e-mail address of the participant input in the host terminal 20, associating it to the conference ID. The controller 11 sends a conference invitation notification to the participant via the communication unit 12 (Step S19). Specifically, the controller 11 obtains the e-mail address corresponding to the participant from the conference management table T2 and sends the conference invitation notification to the obtained e-mail address. The date and time of the conference, the host, the object of the conference, etc. are included in the conference invitation notification.

The participant checks the details of the conference in receipt of the conference invitation notification in the participant terminal 30. The participant accesses the conference management server 10 according to the link in the e-mail and applies for the participation in the conference by the operation via the operation unit 32 (Step S20).

In the conference management server 10, the controller 11 issues a participant ID (user ID) and password to a participant who applied for participation (Step S21). The controller 11 stores the issued participant ID and password in the conference management table T2, associating them to the conference ID.

The controller 11 notifies the participant terminal 30 of the issued participant ID and password via the communication unit 12 (Step S22).

Next, in the host terminal 20, as the host presses the application setting button B12 on the conference setup screen 232 by the operation via the operation unit 22, the application setting screen is displayed on the display 23. As the host selects the application to be used in the conference by the operation via the operation unit 22 (Step S23), the controller 21 sends the information indicating the selected application to the conference management server 10 via the communication unit 24.

In the conference management server 10, the controller 11 sets the application selected in the host terminal 20 as the application to be used in the conference (Step S24). Specifically, the controller 11 stores the name of the selected application in the conference management table T2, associating it to the conference ID. Further, the controller 11 may determine whether or not the personal information is to be shown in the selected application.

Next, the controller 11 generates the personal work area 143 for each participant in the RAM 14 (Step S25). Specifically, the controller 11 generates the personal work area 143 where each participant uses the selected application during the conference.

Next, in the host terminal 20, as the host presses the material registration button B13 on the conference setup screen 232 by the operation via the operation unit 22, the material registration screen is displayed on the display 23. The host selects the material(s) to be used in the conference and determines the authorization setting for the material by the operation via the operation unit 22 (Step S26). The controller 21 sends the selected material and the authorization setting to the conference management server 10 via the communication unit 24.

In the conference management server 10, the controller 11 registers the material selected in the host terminal 20 (Step S27). Specifically, the controller 11 stores the material sent from the host terminal 20 in the storage 15, and also stores the file name of the material and the authorization setting for the file (downloadable or not) in the conference management table T2, associating them to the conference ID.

Next, in the host terminal 20, the host sets the operation authorization and the available period for the conference by the operation via the operation unit 22 (Step S28). For example, as the host inputs the available period to the available period input area 232B on the conference management screen 232, the controller 21 sends the input available period to the conference management server 10 via the communication unit 24.

In the conference management server 10, the controller 11 registers the operation authorization and the available period set in the host terminal 20 (Step S29). Specifically, the controller 11 stores the set operation authorization and the available period in the conference management table T2, associating them to the conference ID.

The conference setup process is completed here.

The process at Steps S20 to S22 is executed at different timings for each participant. The process at Steps S20 to S22 is to be executed at least after the process at Step S19 and may be executed after the process at Steps S23 to S29.

In the conference setup process shown in FIG. 7, the participant apply for participation in response to the conference invitation notification. However, there may be a participation application screen through which anyone can apply for participation.

### Conference Organizing Process

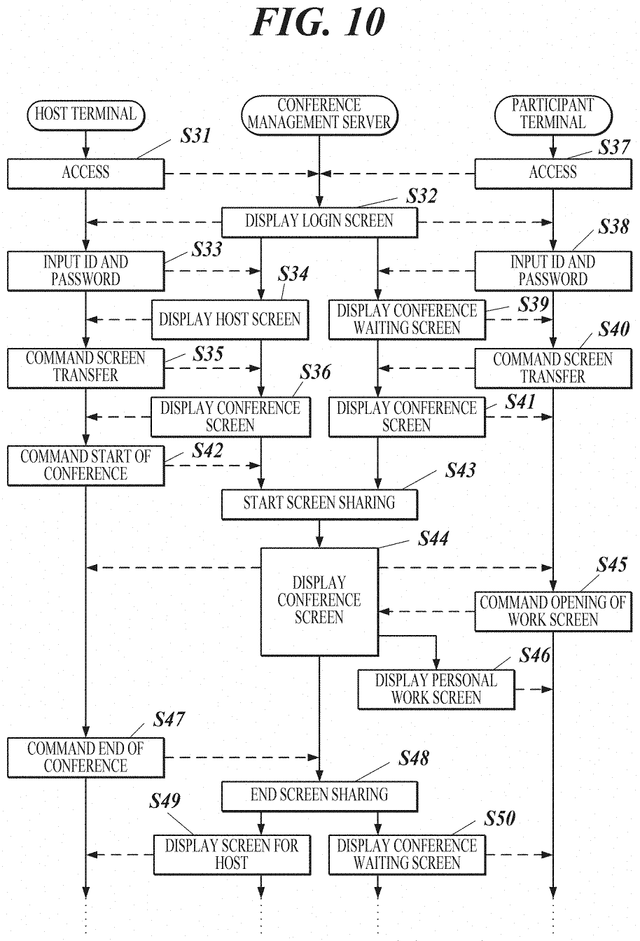

FIG. 10 is a ladder chart showing a conference organizing process executed in the conference system 100. The conference organizing process is executed at the date and time of the conference.

First, as the host accesses the conference management server 10 on the web browser by the operation via the operation unit 22 in the host terminal 20 (Step S31), the controller 11 sends the data for displaying the login screen to the host terminal 20 via the communication unit 12 in the conference management server 10 (Step S32).

The login screen is displayed on the display 23 in the host terminal 20. As the host inputs the user ID and password on the login screen by the operation via the operation unit 22 (Step S33), the controller 21 sends the input user ID and password to the conference management server 10 via the communication unit 24.

In the conference management server 10, as the user ID and password are received via the communication unit 12, the controller 11 executes the user authentication. The user authentication of the host is the same as that at Step S13 in the conference setting process (see FIG. 7).

If the input user ID matches a host ID, the controller 11 sends the data for displaying the host screen to the host terminal 20 via the communication unit 12 (Step S34).

The host screen is displayed on the display 23 in the host terminal 20 (see FIG. 8). As the host commands the screen transfer on the host screen by the operation via the operation unit 22 (Step S35), the controller 11 sends the data for displaying the conference screen to the host terminal 20 via the communication unit 12 (Step S36).

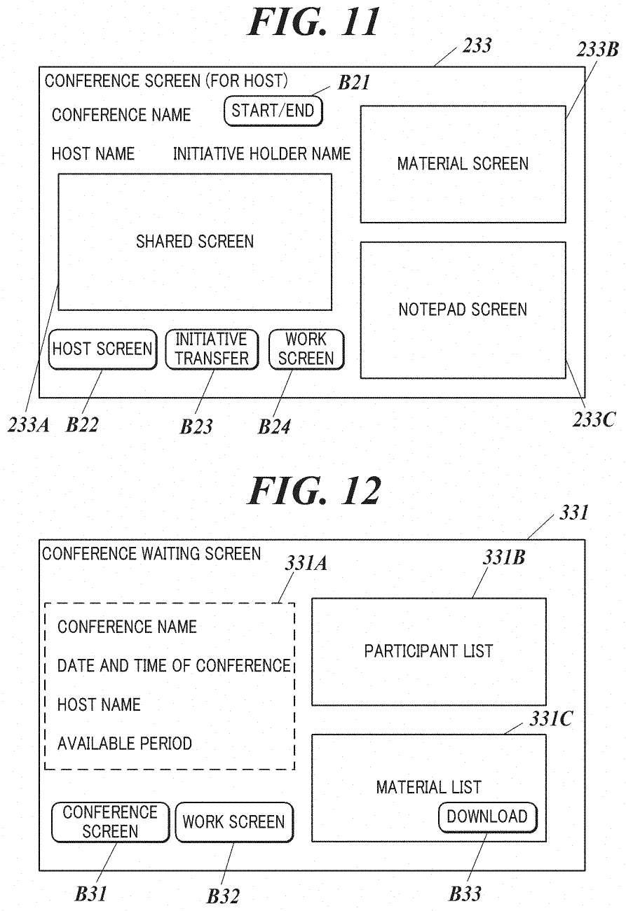

Specifically, as the host presses the conference screen button B1 on the host screen 231 shown in FIG. 8, the conference screen 233 shown in FIG. 11 is displayed on the display 23. The conference screen 233 includes a shared screen 233A, a material screen 233B, a notepad screen 233C, a start/end button B21, a host screen button B22, an initiative transfer button B23, a work screen button B24, etc.

The shared screen 233A is a screen common to multiple terminal devices (host terminal(s) 20, participant terminal(s) 30). The main material or the movie capturing the presenter may be shown on the shared screen 233A.

The material screen 233B is a screen for displaying the content of the material.

The notepad screen 233C is a screen where the host inputs a note, etc.

The start/end button B21 is a button for a command of start or end of the conference.

The host screen button B22 is a button for a command of transfer to the host screen (see FIG. 8).

The initiative transfer button B23 is a button for requesting of the initiative transfer.

The work screen button B24 is a button for a command of transfer to the personal work screen.

As the conference screen is displayed on the display 23 in the host terminal 20, the host may be one of the participants.

On the other hand, as the participant accesses the conference management server 10 on the web browser by the operation via the operation unit 32 in the participant terminal 30 (Step S37), the controller 11 sends the data for displaying the login screen to the participant terminal 30 via the communication unit 12 in the conference management server 10 (Step S32).

The login screen is displayed on the display 33 in the participant terminal 30. As the participant inputs the user ID and password on the login screen by the operation via the operation unit 32 (Step S38), the controller 31 sends the input user ID and password to the conference management server 10 via the communication unit 34.

As the user ID and password are received via the communication unit 12 in the conference management server 10, the controller 11 executes the user authentication. Specifically, the controller 11 retrieves the record corresponding to the user ID input in the participant terminal 30 from the participant information in the conference management table T2 in the storage 15. Then, if the password included in the retrieved record matches the password input in the participant terminal 30, the controller 11 determines that the user authentication as a participant is successfully executed.

If the input user ID is a participant ID, the controller 11 sends the data for displaying the conference waiting screen to the participant terminal 30 via the communication unit 12 (Step S39).

The conference waiting screen is displayed on the display 33 in the participant terminal 30.

FIG. 12 shows an example of the conference waiting screen 331 displayed on the display 33. The conference waiting screen 331 includes a conference information display area 331A, a participant list display area 331B, a material list display area 331C, a conference screen button B31, a work screen button B32, a download button B33, etc.

Information such as the conference name, the date and time of conference, the name of host, and the available period is shown in the conference information display area 331A.

The list of participants in the conference is shown in the participant list display area 331B.

The list of materials set for the conference is shown in the material lists display area 331C. The authorization setting (downloadable or not) for each material is also shown in the material list.

The conference screen button B31 is a button for a command of transfer to the conference screen.

The work screen button B32 is a button for a command of transfer to the personal work screen.

The download button B33 is a button for a command of downloading of the material. The participant may download the material which he/she is allowed to download by pressing the download button B33. On the other hand, the material which the participant is not permitted to download may only be shown on the web screen. Even in that case, the material may be handled with the application which is a function of the conference management server 10 (details to be described later).

As the participant commands the screen transfer on the conference waiting screen by the operation via the operation unit 32 (Step S40), the controller 11 sends the data for displaying the conference screen to the participant terminal 30 via the communication unit 12 (Step S41).

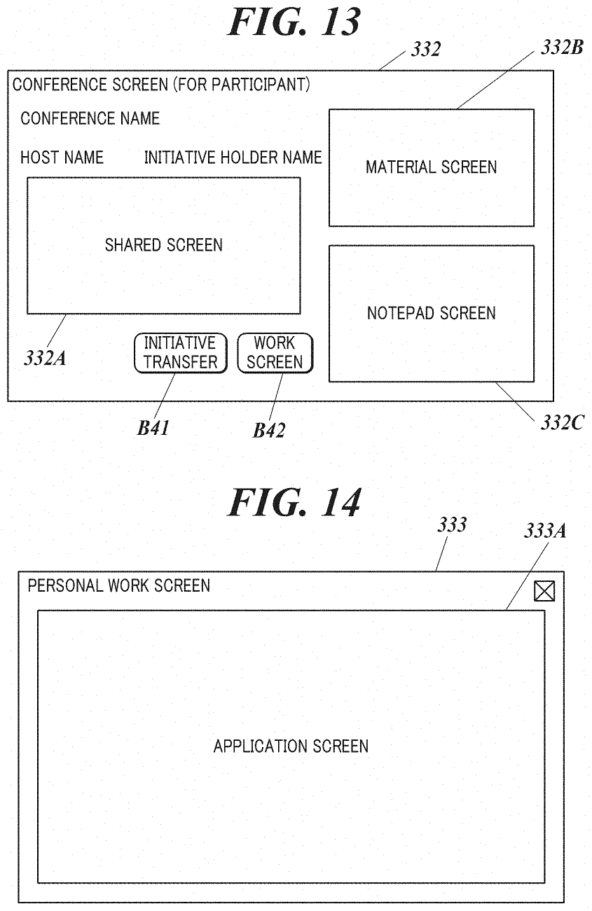

Specifically, as the participant presses the conference screen button B31 on the conference waiting screen 331 shown in FIG. 12, the conference screen 332 shown in FIG. 13 is displayed on the display 33. The conference screen 332 includes a shared screen 332A, a material screen 332B, a notepad screen 332C, an initiative transfer button B41, a work screen button B42, etc.

The shared screen 332A is a screen common to multiple terminal devices (host terminal(s) 20, participant terminal(s) 30).

The material screen 332B is a screen for displaying the content of the material.

The notepad screen 332C is a screen where the participant inputs a note, etc.

The initiative transfer button B41 is a button for requesting of the initiative transfer.

The work screen button B42 is a button for a command of transfer to the personal work screen.

As the host commands the start of the conference by the operation via the operation unit 22 in the host terminal 20 (Step S42), the screen sharing starts in the host terminal 20 and the participant terminals 30 (Step S43). Specifically, as the host presses the start/end button B21 on the conference screen 233 shown in FIG. 11, the screen sharing starts.

The conference screen is displayed in the host terminal 20 and the participant terminals 30, where the conference screen includes the shared screen common in the host terminal 20 and the participant terminals 30 (Step S44). That is, the shared screen 233A of the conference screen 233 (see FIG. 11) displayed on the display 23 of the host terminal 20 has the same content as the shared screen 332A of the conference screen 332 (see FIG. 13) displayed on the display 33 of each participant terminal 30. The controller 11 of the conference management server 10 uses the shared work area 142 in the conference work area 141 of the target conference as the work area for displaying the shared screen 233A and the shared screen 332A.

As the participant commands the opening of the work screen on the conference screen by the operation via the operation unit 32 in the participant terminal 30 (Step S45), the controller 11 sends the data for displaying the personal work screen to the participant terminal 30 via the communication unit 12 (Step S46).

Specifically, as the participant presses the work screen button B42 on the conference screen 332 shown in FIG. 13, the personal work screen 333 shown in FIG. 14 is displayed on the display 33. The controller 11 of the conference management server 10 uses the personal work area 143 prepared for each participant in the conference work area 141 of the target conference as the work area for displaying the personal work screen 333. The personal work screen 333 includes an application screen 333A. The application which is set beforehand for the conference is shown on the application screen 333A with the medical information such as medical images (material(s) predetermined for the conference) to be used in the application being inserted. The controller 11 refers to the conference management table T2, and, if the personal information is to be hidden in the personal information display/hide setting of the application information of the target conference, the personal information is deleted from the medical information used in the application so that the personal information such as a patient's name, date of birth, and address is not shown on the application screen 333A. Alternatively, the content including the personal information may be replaced by ciphers.

The controller 11 provides processing results of the medical information (registered material) processed by the predetermined application for the participant terminals 30, using the personal work area 143 of the participant who uses the concerning participant terminal 30, according to the operation command from the participant terminal 30 used by the participant. The controller 11 may automatically set the material for the conference as the target of processing of the application. Alternatively, the controller 11 may set the material which the user selects from the materials for the conference as the target of processing of the application. The participant may handle the material using the application suitably set for the conference on the personal work screen 333 (application screen 333A). The handling of the material includes display of medical image, image adjustment such as adjustment of density, enlargement, and reduction, measurement, addition of annotation, etc.

Concerning the handling of the material, as the participant in the conference may handle the material in the same way as the presenter actually does in an online conference (for example, gradation processing of an X-ray image for a clearer observation of a lesion, etc.), the participant may deepen the understanding. This is effective for the improvement of learning efficiency in an actual conference, allowing the use of the materials which the participants may not easily handle locally, while the confidentiality is maintained concerning highly confidential materials such as medical images and medical information.

In a case where multiple application are set, the application screens for all the set applications may be displayed, or the applications may be switched using tabs, etc.

As the host commands the end of conference by the operation via the operation unit 22 in the host terminal 20 (Step S47), the screen sharing in the host terminal 20 and the participant terminals 30 ends (Step S48). Specifically, as the host presses the start/end button B21 on the conference screen 233 shown in FIG. 11, the screen sharing ends.

The host screen 231 (see FIG. 8) is displayed on the display 23 in the host terminal 20 (Step S49), and the conference waiting screen 331 (see FIG. 12) is displayed on the display 33 in the participant terminal 30 (Step S50).

The conference organizing process is completed here.

### Initiative Transfer Process

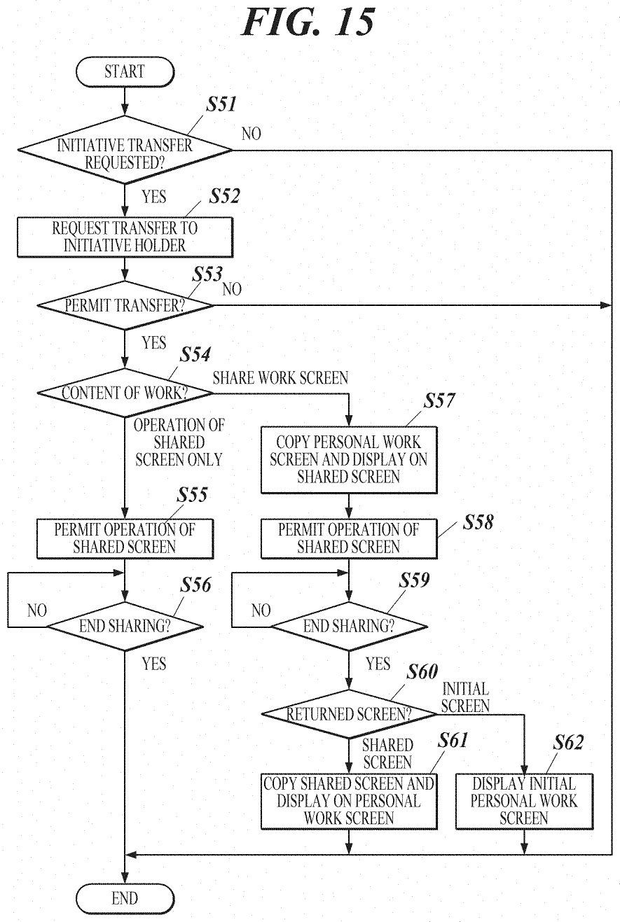

FIG. 15 is a flowchart showing an initiative transfer process executed in the conference management server 10. The initiative transfer process is a process executed during the conference (Steps S43 to S47 in the conference organizing process shown in FIG. 10).

First, the controller 11 determines whether or not one of the participant terminals 30 requests the initiative transfer (Step S51). Specifically, the controller 11 determines whether or not the initiative transfer button B41 is pressed on the conference screen 332 (see FIG. 13) displayed on the display 33 of the participant terminal 30.

If one of the participant terminals 30 requests the initiative transfer (Step S51; YES), the controller 11 sends a request for initiative transfer to the initiative holder (Step S52). The initiative holder is an individual who has the operation authorization for the shared screen, and the host of the conference or the presenter is designated as the initiative holder, for example.

Next, the controller 11 determines whether or not the initiative holder permits the transfer of the initiative (Step S53).

If the initiative holder permits the transfer of the initiative (Step S53; YES), the controller 11 prompts the participant who requested the initiative transfer to select the content of work (Step S54). Specifically, the controller 11 sends, to the participant terminal 30 used by the participant who requested the initiative transfer, the display data for selection of either or both of the operation of the shared screen and the sharing of the work screen as the content of work via the communication unit 12.

If the participant requests the initiative for “the operation of the shared screen” only (Step S54; the operation of the shared screen only), the controller 11 permits the participant who requested the initiative transfer to operate the shared screen (Step S55). The participant who is permitted the initiative transfer has the operation authorization for the shared screen 332A on the conference screen 332 (see FIG. 13) displayed on the display 33 in the participant terminal 30.

Then, if the participant holding the initiative does not command the end of sharing (Step S56; NO), the process is back to Step S56.

If the participant requests the initiative in “the sharing of the work screen” at Step S54 (Step S54; sharing of the work screen), the controller 11 copies the personal work screen associated to the participant who requested the initiative transfer and the copied personal work screen is shown on the shared screen of the participant terminals 30 used by all the participants (Step S57). That is, the personal work screen shown in the participant terminal 30 of the participant who requested the initiative transfer is shown on the shared screen common to the participant terminals 30 used by all the participant by the controller 11. The controller 11 permits the participant who requested the initiative transfer to operate the shared screen (Step S58).

After Step S58, if the participant holding the initiative commanded the end of sharing (Step S59; YES), the controller 11 prompts the participant to select the returned screen (Step S60). Specifically, the controller 11 sends, to the participant terminal 30 used by the participant who released the initiative, the display data for prompting the participant to select either the shared screen or the initial screen as the returned screen via the communication unit 12.

If the shared screen is selected as the returned screen (Step S60; shared screen), the controller 11 copies the shared screen and displays it on the personal work screen of the participant terminal 30 used by the participant who released the initiative (Step S61).

If the initial screen is selected as the returned screen at Step S60 (Step S60; initial screen), the personal work screen which is copied at Step S57 is shown on the personal work screen of the participant terminal 30 used by the participant who released the initiative by the controller 11 (Step S62).

The initiative transfer process is completed at the following timings; when no initiative transfer is requested from any participant terminal 30 at Step S51 (Step S51; NO); when the initiative holder does not permit the initiative transfer at Step S53 (Step S53; NO); when the participant holding the initiative commands the end of sharing at Step S56 (Step S56; YES), after Step S61; and after Step S62.

### Post-Conference Process (During Available Period)

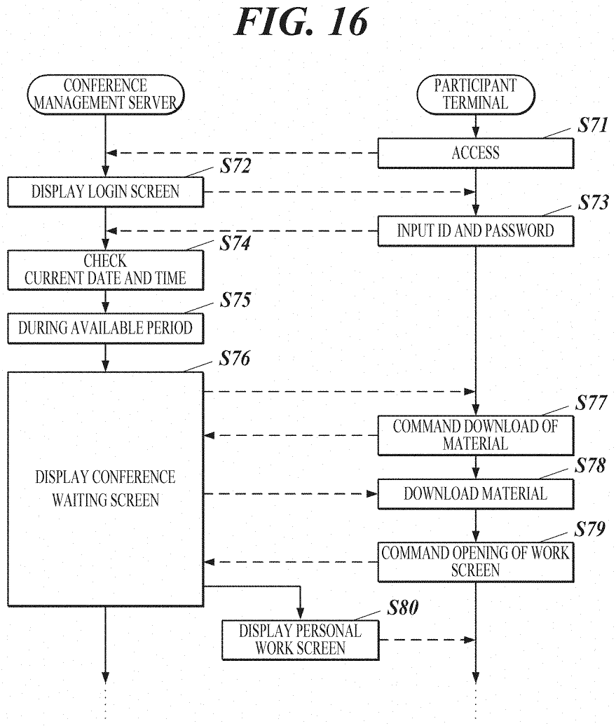

FIG. 16 is a ladder chart showing a post-conference process (during the available period) executed in the conference system 100. The post-conference process is a process to be executed after the conference ends.

First, as the participant accesses the conference management server 10 on the web browser by the operation via the operation unit 32 in the participant terminal 30 (Step S71), the controller 11 sends the data for displaying the login screen to the participant terminal 30 via the communication unit 12 in the conference management server 10 (Step S72).

The login screen is displayed on the display 33 in the participant terminal 30. As the participant inputs the user ID and password on the login screen by the operation via the operation unit 32 (Step S73), the controller 31 sends the input user ID and password to the conference management server 10 via the communication unit 34.

In the conference management server 10, as the user ID and password are received via the communication unit 12, the controller 11 executes the user authentication. The user authentication is the same as that at Step S38 in the conference organizing process (see FIG. 10).

If the input user ID is a participant ID, the controller 11 checks the current date and time (Step S74) and determines whether or not it is during the available period. Specifically, the controller 11 obtains the current date and time from the timing unit 13, refers to the conference management table T2 in the storage 15, reads out the available period associated to the participant ID input from the participant terminal 30, and determines whether or not the current date and time is during the available period.

If it is during the available period (Step S75), the controller 11 sends the data for displaying the conference waiting screen to the participant terminal 30 via the communication unit 12 (Step S76).

The conference waiting screen (see FIG. 12) is displayed on the display 33 in the participant terminal 30. As the participant selects the material which is set as downloadable on the conference waiting screen and commands downloading of the selected material by the operation via the operation unit 32 (Step S77), the selected material is downloaded (Step S78). Specifically, as the participant presses the download button B33 on the conference waiting screen 331 shown in FIG. 12, the selected material is obtained from the conference management server 10 and stored in the storage 36 of the participant terminal 30. The process at Step S77 and S78 is to be executed as necessary.

In the participant terminal 30, as the participant commands the opening of the work screen on the conference waiting screen by the operation via the operation unit 32 (Step S79), the controller 11 sends the data for displaying the personal work screen to the participant terminal 30 via the communication unit 12 (Step S80).

Specifically, as the participant presses the work screen button B32 on the conference waiting screen 331 shown in FIG. 12, the personal work screen 333 shown in FIG. 14 is displayed on the display 33. In that way, the participant may view the medical information or edit the medical images on the personal work screen 333 which is provided by the conference management server 10 using the personal work area 143 for each participant during the available period even after the conference ends.

The post-conference process (during the available period) is completed here.

### Post-Conference Process (Outside Available Period)

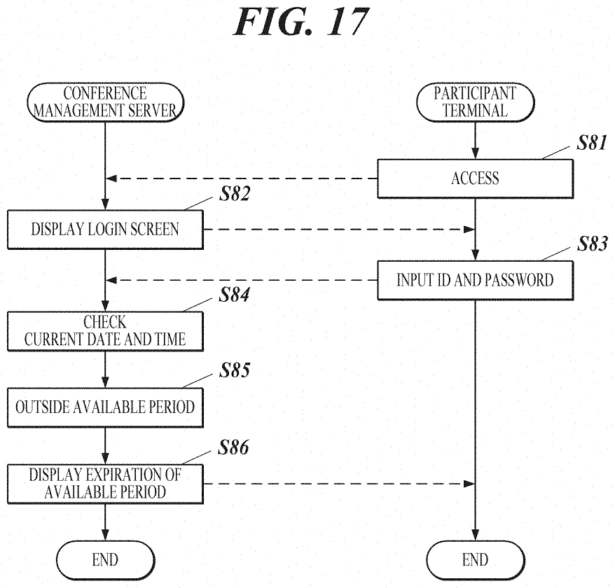

FIG. 17 is a ladder chart showing a post-conference process (outside the available period) executed in the conference system 100.

The process at Steps S81 to S84 is not described, as it is the same as the process at Steps S71 to S74 of the post-conference process (during the available period) shown in FIG. 16.

If the checked current date and time is outside the available period (Step S85), the controller 11 sends the data for displaying a message that it is outside the available period to the participant terminal 30 via the communication unit 12 (Step S86).

The indication that it is outside the available period is displayed on the display 33 in the participant display 30.

Then, the login screen is displayed again in the participant terminal 30 and the post-conference process (outside the available period) is completed.

As described above, according to this embodiment, the processing results of the medical information processed with the predetermined application program are provided for the participant terminal 30 used by each participant, using the personal work area 143 prepared for each participant. Thus, the medical information and the application program do not need to be downloaded beforehand in the participant terminal 30, and the load imposed on the participant terminal 30 may be decreased. Accordingly, the information may be provided for the participant terminal 30 without changes being made to the environment of the terminal device 30 used by the participant.

Specifically, the participant may handle and process the material provided by the conference management server 10 in the participant terminal 30. For example, the participant may view, adjust, measure, and annotate the medical image in the participant terminal 30.

The material provided for each participant is stored in the conference management server 10, and the processing according to the operation command from each participant is executed using the personal work area 143 in the conference management server 10. Thus, the participant does not need to install the application program or download the material beforehand. Each participant may handle the material provided by the conference management server 10 on his/her own, and the work efficiency is improved.

Concerning the participant who is permitted the sharing of the personal work screen, the personal work screen displayed in the participant terminal 30 used by the participant may be displayed on the shared screen which is common in multiple terminal devices (participant terminal(s) 30, host terminal(s) 20). In that way, the personal work screen which is processed using the personal work area 143 may be published on the shared screen.

As the available period is set for the personal work area 143, the processing results of the medial information processed with the predetermined application program may be provided for each participant terminal 30 using the personal work area 143 during the available period even after the conference ends.

The personal information may be shown or hidden as set beforehand for each application for the conference. Thus, the security against information leaks may be strengthened concerning the medical images including personal information of patient.

The embodiment described above is merely an example of the information processing server according to the present invention, and is not intended to limit the invention. The detailed configuration or operation may be suitably modified without departing from the scope of the invention.

For example, the shared screen 332A (see FIG. 13) and the personal work screen 333 (see FIG. 14) are displayed in different windows in the participant terminal 30 in the embodiment described above. However, the shared screen 332A and the personal work screen 333 may be displayed in the same window.

The personal work screen is displayed in the participant terminal 30 in the embodiment described above. However, the personal work screen may be displayed in the host terminal 20, the host being one of the participants.

The host terminal 20 and the participant terminal 30 may include a voice input/output unit such as a microphone and a speaker, and the voice of the participant received by a terminal device may be output to the terminal devices.

A portable recording/storage medium such as a CD-ROM may be used as a computer-readable medium storing the programs for executing various kinds of processing therein, besides an HDD and a non-volatile semiconductor memory. Further, a carrier wave may be used as a medium to provide data of the programs via communication network.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-129844, filed on Jul. 9, 2018, is incorporated herein by reference in its entirety.

What is claimed is:

1. An information processing server to which terminal devices used by respective participants are connected for data communication and which provides the terminal devices with medical information including a medical image, the information processing server comprising:
a memory which includes a shared work area for displaying a shared screen which is commonly shown in the terminal devices and personal work areas each for displaying a personal work screen in each of the terminal devices used by the respective participants, the work areas being prepared for the respective participants; and
a hardware processor which provides, according to an operation command from the each of the terminal devices used by the respective participants, the each of the terminal devices used by the respective participants with a processing result of the medical information processed with a predetermined application program, using the personal work areas for the respective participants using the respective terminal devices.

2. The information processing server according to claim 1,
wherein the medical information to be processed with the application program is the medical image, and
wherein the application program is a program for executing display processing, image adjustment processing, measurement processing, or annotation addition processing on the medical image.

3. The information processing server according to claim 1, wherein the hardware processor causes the personal work screen displayed in a terminal device used by a predetermined participant out of the multiple participants to be displayed on the shared screen which is commonly displayed in the multiple terminal devices.

4. The information processing server according to claim 1,
wherein the hardware processor sets an available period of the personal work area, and
the personal work area is available during the available period.

5. A non-transitory computer-readable recording medium storing a program causing a computer which controls an information processing server to which terminal devices used by respective participants are connected for data communication and which provides the terminal devices with medical information including a medical image to perform:
preparing, in a memory, a shared work area for displaying a shared screen which is commonly shown in the terminal devices and personal work areas each for displaying a personal work screen in each of the terminal devices used by the respective participants, the work areas being prepared for the respective participants; and
providing, according to an operation command from the each of the terminal devices used by the respective participants, the each of the terminal devices used by the respective participants with a processing result of the medical information processed with a predetermined application program, using the personal work areas for the respective participants using the respective terminal devices.
